(19) United States
(12) Patent Application Publication
(10) Pub. No.: US 2026/0251340 A1
(43) Pub. Date: Aug. 27, 2026

(54) METHOD AND COANDA-EFFECT APPARATUS FOR ENHANCED UPPER-AIR UV-C AIR SANITATION

(71) Applicant: Thomas Dunbar, Dundee, NY (US)

(72) Inventor: Thomas Dunbar, Dundee, NY (US)

(21) Appl. No.: 19/550,720

(22) Filed: Feb. 26, 2026

Related U.S. Application Data
(60) Provisional application No. 63/763,469, filed on Feb. 26, 2025.

(51) Int. Cl. F24F 13/08; A61L 9/20; F24F 8/22

(52) U.S. Cl. CPC F24F 13/081; A61L 9/20; F24F 8/22; A61L 2209/12; A61L 2209/16; F24F 2221/28

(57) ABSTRACT

A system for enhancing ultraviolet air disinfection in an enclosed space includes at least one Coandă-effect diffuser coupled to an HVAC supply and mounted adjacent the ceiling of the enclosed space. The diffuser redirects supplied air received from the HVAC supply along the ceiling to induce a room-scale rotational airflow pattern. At least one upper-air ultraviolet luminaire is positioned within the upper region of the enclosed space to generate an ultraviolet irradiation region. The diffuser establishes a circulating airflow that entrains contaminated air from the lower region and transports the contaminated air into the ultraviolet irradiation region produced by the upper-air ultraviolet luminaire. The circulating airflow delivers treated air from the ultraviolet irradiation region toward an occupied region of the enclosed space.

### CROSS REFERENCE TO RELATED APPLICATIONS

This application claims the benefit of U.S. Provisional Application No. 63/763,469 filed on Feb. 26, 2025, and entitled, METHOD AND COANDA-EFFECT APPARATUS FOR ENHANCED UPPER-AIR UV-C AIR SANITATION, the entirety of which is hereby incorporated by reference.

### FIELD OF THE INVENTION

The present invention relates generally to indoor air sanitation and pathogen reduction systems, and more particularly to apparatuses and methods for enhancing upper-air ultraviolet (UV) air disinfection through controlled room-scale airflow. Still more particularly, the invention relates to Coandă-effect diffuser systems integrated with building HVAC infrastructure to establish rotational airflow patterns that transport contaminated air into upper-air ultraviolet treatment regions and deliver treated air back toward occupied zones.

### BACKGROUND OF THE INVENTION

Airborne transmission of pathogens in enclosed environments presents ongoing challenges in healthcare, residential, commercial, and public spaces. Upper-air ultraviolet germicidal irradiation (UVGI) systems are commonly employed in hospitals and similar facilities to inactivate airborne pathogens while maintaining occupant safety by confining ultraviolet radiation to regions above head height.

Conventional upper-air UVGI installations rely primarily on passive mechanisms—such as thermal plumes generated by occupants or incidental HVAC mixing—to transport contaminated air into the ultraviolet treatment zone. In practice, these mechanisms are often unreliable. Limited airflow near floor level, interference from HVAC diffusers, turbulent mixing, and drag forces acting on aerosolized particles can significantly reduce the likelihood that pathogens will consistently enter the ultraviolet field. As a result, residence time and exposure dosage are frequently insufficient for dependable pathogen inactivation.

The inventor's prior work disclosed active air sanitization devices that draw air from lower regions of a room, process it through an internal sanitization mechanism, and discharge treated air near the ceiling, thereby creating a rolling airflow column within the space. While such active devices can be effective, their deployment requires dedicated powered equipment within the room.

Accordingly, there remains a need for systems that can leverage existing HVAC airflow to actively organize room-scale air movement, reliably transport contaminated air into upper-air ultraviolet regions, and deliver cleaner air back to occupants—without requiring standalone powered air sanitization units.

There is further a need for retrofit-compatible solutions that integrate with standard HVAC supply ducts, enhance the effectiveness of existing upper-air UV installations, and adapt to varying room geometries, occupant placement, and return-air configurations.

### BRIEF SUMMARY OF THE INVENTION

The present invention provides systems and methods for enhancing upper-air ultraviolet air sanitization by establishing a controlled rotational airflow pattern within an enclosed space using a Coandă-effect diffuser coupled to an HVAC supply.

In one aspect, conditioned air supplied by an HVAC system is redirected along a ceiling by a Coandă-effect diffuser to form a ceiling-attached horizontal jet. This jet initiates a room-scale rolling airflow column in which air travels across the ceiling, descends along a first wall, sweeps across the floor, rises along an opposite wall, and returns through an upper-air ultraviolet irradiation region. Through this circulation, contaminated lower-room air is repeatedly transported into the ultraviolet field for inactivation, while progressively cleaner air is delivered back toward occupied regions.

Unlike prior approaches relying on passive thermal plumes or localized purification devices, the present invention utilizes the building HVAC system itself as the motive force for organized room-scale circulation, thereby enhancing ultraviolet exposure efficiency without substantial additional energy consumption.

In preferred embodiments, diffuser outlet apertures are aligned substantially parallel to emission apertures of upper-air ultraviolet luminaires to promote coordinated airflow and ultraviolet interaction. Diffuser geometry may be configured to preserve airflow velocity while redirecting flow through large directional changes with minimal pressure loss.

In further embodiments, diffuser orientation and placement may be selected based on computational fluid dynamics modeling accounting for room geometry, occupant locations, HVAC supply placement, and return grille placement, enabling optimized airflow patterns across a wide range of architectural environments.

The invention is applicable to healthcare rooms, including multi-occupant hospital rooms, as well as other enclosed spaces such as clinics, medical examination rooms, classrooms, conference rooms, auditoriums, offices, long-term care facilities, and residential environments. Any illustrated room configuration is provided solely as an exemplary embodiment and is not intended to limit the scope of application.

In an alternative embodiment, an airflow-integrated ultraviolet luminaire is provided, configured to receive circulating ceiling-attached airflow directly into an internal ultraviolet exposure chamber prior to re-entry into the room, further increasing pathogen inactivation efficiency.

Collectively, the disclosed systems provide a retrofit-compatible, HVAC-integrated approach to actively transporting airborne pathogens into upper-air ultraviolet treatment zones, achieving repeated ultraviolet exposure while simultaneously delivering treated air back toward occupants, thereby reducing airborne infection risk and improving indoor air quality. Throughout this disclosure, the Coandă-effect diffuser assembly may be referred to as a “coWANduc,” representing an HVAC-integrated airflow-directing device configured to establish controlled room-scale circulation for enhanced air sanitation.

The present invention is distinct from the inventor's previously issued air sanitization systems, including U.S. Pat. No. 11,357,882 and its continuation U.S. Pat. No. 12,343,459, which disclose standalone, actively powered air treatment devices that draw room air into an internal sanitization chamber and discharge treated air back into the space. In contrast, the systems and methods disclosed herein do not rely on a dedicated in-room air processing appliance to generate airflow. Rather, the present invention utilizes existing building HVAC supply airflow, reshaped by a Coandă-effect diffuser, to establish a room-scale rotational circulation pattern that transports contaminated air into upper-air ultraviolet regions and returns treated air toward occupied zones. This architectural, HVAC-integrated approach represents a fundamentally different mechanism of action, shifting from device-driven airflow generation to diffuser-mediated airflow orchestration using existing building infrastructure.

The embodiments illustrated herein, including the example of a two-occupant hospital room, are provided solely for purposes of explanation and clarity and are not intended to limit the scope of the invention. The disclosed systems and methods are equally applicable to a wide range of enclosed environments, including but not limited to single-patient hospital rooms, medical examination rooms, clinics, classrooms, conference rooms, auditoriums, offices, long-term care facilities, and other occupied indoor spaces where enhanced airborne pathogen control and improved indoor air quality are desired.

Diffuser placement, orientation, and airflow characteristics may be adapted for each application based on room geometry, occupant locations, HVAC supply and return placement, and computational fluid dynamics modeling, enabling tailored airflow patterns for diverse architectural layouts and use cases.

The disclosed systems are inherently scalable and may be implemented using a single diffuser in smaller rooms or multiple coWANduc diffuser assemblies distributed across larger spaces. In multi-diffuser configurations, coordinated airflow patterns may be established across zones or throughout entire rooms, corridors, or open areas such as auditoriums and conference halls. Diffusers may be arranged to operate cooperatively, enabling room-scale or building-scale circulation patterns while utilizing existing HVAC infrastructure. Such scalability supports deployment in a wide range of environments, from individual patient rooms to large public or commercial spaces, without requiring fundamental changes to the underlying airflow control methodology.

In addition to enhancing pathogen inactivation, the disclosed HVAC-integrated airflow system provides significant potential energy and operational benefits. By actively circulating and sanitizing air that has already been conditioned within the space, the invention reduces reliance on high volumes of energy-intensive outside air to achieve indoor air quality objectives. Improved in-room air sanitation can therefore support reduced ventilation rates while maintaining or improving occupant health outcomes, leading to lower heating, cooling, and humidity-control loads.

In healthcare environments, enhanced airborne pathogen reduction may also contribute to decreased rates of hospital-acquired infections and shorter patient stays, yielding both clinical and economic benefits. Similar advantages apply to other occupied spaces, where improved air quality can reduce absenteeism, improve occupant comfort, and lower overall building energy consumption. Accordingly, the present invention supports both infection control objectives and broader sustainability and operational efficiency goals.

### BRIEF DESCRIPTION OF THE DRAWINGS

FIG. 1 is a perspective view of a Coandă-effect diffuser assembly according to an embodiment of the present invention.

FIGS. 2A and 2B are side and sectional views of the diffuser assembly of FIG. 1.

FIG. 3 illustrates internal equal-area flow sections of the diffuser showing preservation of airflow velocity between inlet and outlet.

FIG. 4 illustrates directional redirection of airflow through the diffuser.

FIGS. 5A and 5B depict an alternate angular configuration between diffuser and ceiling.

FIGS. 6A and 6B depict an exemplary prior-art upper-air ultraviolet luminaire, with FIG. 6A showing internal components.

FIG. 7 illustrates an ultraviolet radiation emission pattern produced by an upper-air luminaire.

FIG. 8 is a perspective view of an exemplary room showing placement of diffuser assemblies and upper-air ultraviolet luminaires.

FIG. 9 is a side elevation view of the room of FIG. 7.

FIG. 10 is a top plan view illustrating ultraviolet radiation coverage within the room.

FIG. 11 is a perspective view illustrating diffuser placement relative to luminaries.

FIG. 12 is a top plan view illustrating diffuser outlets, occupants, and HVAC return placement.

FIG. 13 is a top plan view illustrating diffuser outlets, occupants, and alternate HVAC return placement.

FIG. 14 illustrates alignment of diffuser outlet apertures along an axis substantially parallel to an axis defined by upper-air luminaires.

FIG. 15 illustrates a conceptual rotating airflow column within the room.

FIG. 16 is a perspective view illustrating three-dimensional airflow circulation.

FIG. 17 illustrates airflow interaction with occupants and surfaces within the room.

FIG. 18 illustrates repeated airflow exposure through ultraviolet radiation regions.

FIG. 19 illustrates combined diffuser-induced airflow circulation and ultraviolet exposure within the room.

FIG. 20 is a perspective view of an airflow-integrated ultraviolet luminaire according to an alternative embodiment.

FIGS. 21A and 21B are end and front views of the airflow-integrated ultraviolet luminaire of FIG. 20.

FIG. 22 is a transparent perspective view of the airflow-integrated ultraviolet luminaire illustrating internal airflow and ultraviolet exposure regions.

FIG. 23 illustrates airflow interaction between the diffuser and airflow-integrated ultraviolet luminaire.

### DETAILED DESCRIPTION

The following detailed description is provided to illustrate representative embodiments of the present invention and is not intended to limit the scope of the invention.

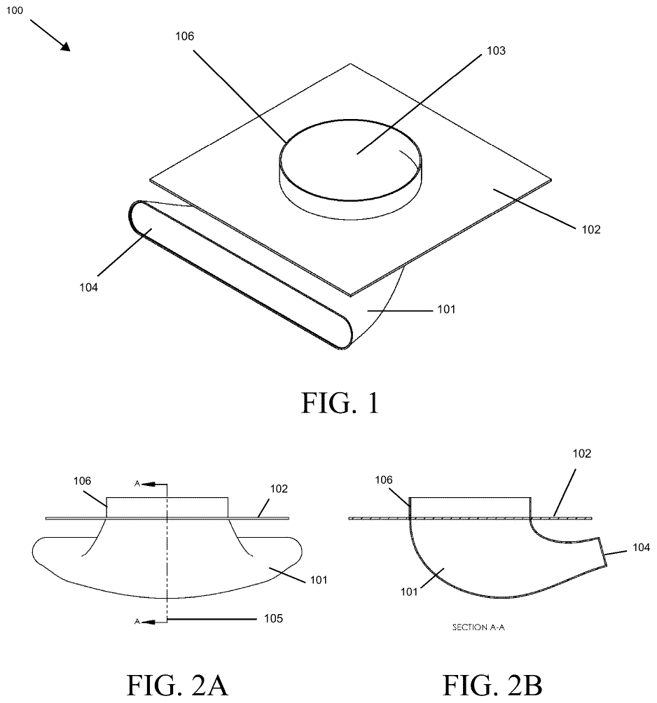

Referring initially to FIG. 1, a Coandă-effect diffuser assembly 100 includes a diffuser body 101 coupled to a ceiling interface plate 102 and an HVAC inlet area 103 via an HVAC interface collar 106. Conditioned air supplied by the building HVAC system enters the diffuser assembly through inlet area 103 and is discharged through diffuser outlet aperture 104.

As shown in FIGS. 2A and 2B, diffuser body 101 defines an internal flow path configured to redirect incoming HVAC airflow while maintaining a low profile suitable for ceiling mounting. Section cut line 105 illustrates a representative cross-section through the diffuser geometry.

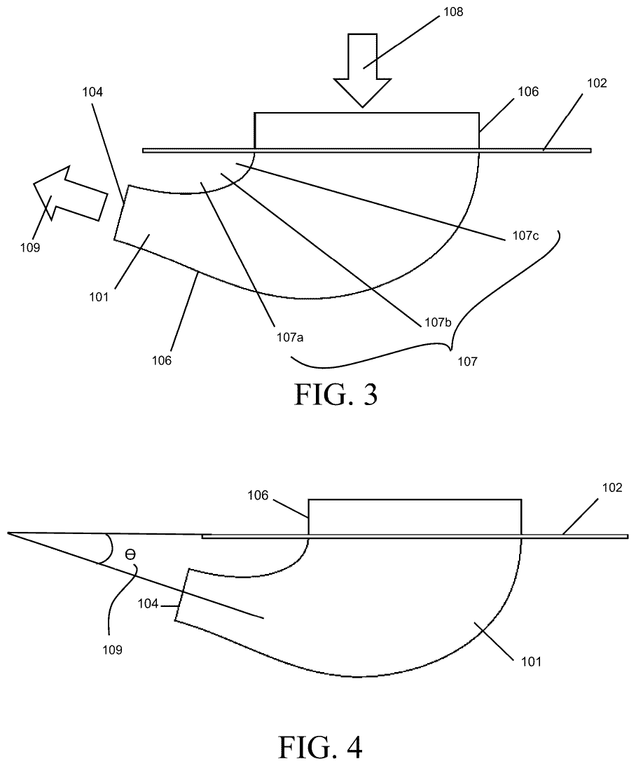

Referring to FIG. 3, the internal geometry of diffuser body 101 forms a plurality of equal cross-sectional flow regions 107a-107c collectively defining equal cross-sectional area 107. Air enters the diffuser as inlet HVAC flow 108 and exits as redirected flow 109. In one embodiment, inlet and outlet cross-sectional areas are substantially equal, such that airflow exits the diffuser at approximately the same average velocity as it enters. An optional UVC antireflection coating 106 may be applied to diffuser surfaces.

FIG. 4 illustrates directional redirection of airflow relative to ceiling interface plate 102, with an average directional change angle 109 imparted by the diffuser geometry. In this manner, the diffuser imposes minimal pressure loss beyond that associated with the change in flow direction. In one aspect of the invention, diffuser body 101 is configured such that diffuser output aperture 104 has an angle 109 which generates the desired Coandă-effect adherence of the discharged air along ceiling 302.

In preferred embodiments the discharge direction may be substantially parallel to the ceiling (i.e., angle 109 is zero degrees plus/minus about five degrees relative to the ceiling plane) or may be selected within a range (for example and without limitation, between about five degrees and about twenty-five degrees, and more preferably between about ten degrees and about twenty degrees) to maintain discharged air attachment while achieving the desired downstream wall impingement and circulation. In exemplary embodiments, the angle may be selected using computational fluid dynamics modeling and/or empirical commissioning for a given installation, and in some embodiments may be adjustable. Thus, the discharge angle that best maintains a ceiling-attached (Coandă-effect) jet and produces the desired room-scale rotational circulation depends on many factors including discharge velocity, outlet geometry (slot height/width), proximity to the ceiling, room geometry, and supply/return placement.

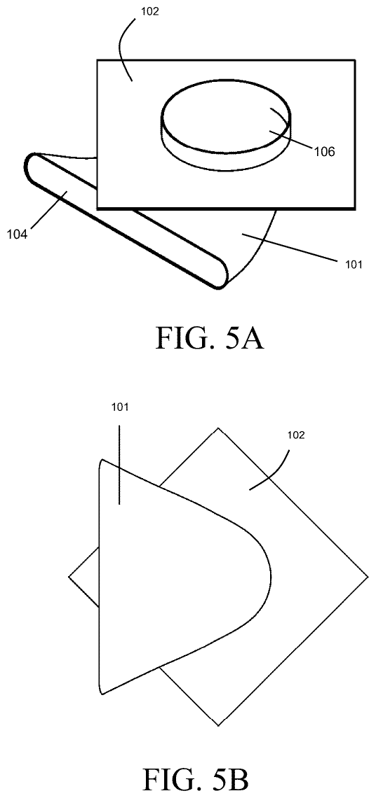

FIGS. 5A and 5B illustrate alternate orientation angle between the diffuser body 101 and ceiling interface plate 102. Fluid dynamic studies may indicate optimum angles between body 101 and ceiling plate 102 in retrofit installations where HVAC supply locations 200, Luminary 500 locations and HVAC return locations 204 are established, as will be described in greater detail below.

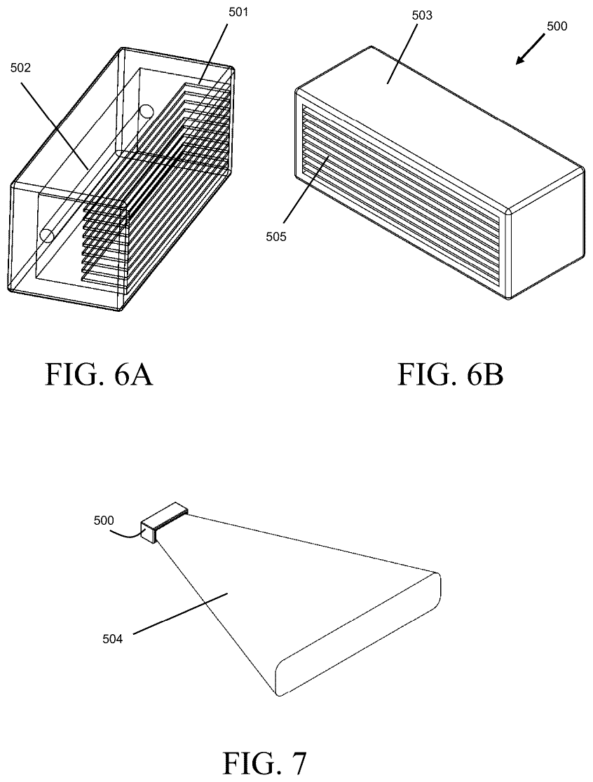

FIGS. 6A and 6B illustrate a representative prior-art upper-air ultraviolet luminaire 500 including ultraviolet source 502, housing 503, luminaire emission apertures 505, and light-angle-reducing baffles 501. FIG. 7 illustrates an exemplary ultraviolet radiation field 504 produced by luminaire 500.

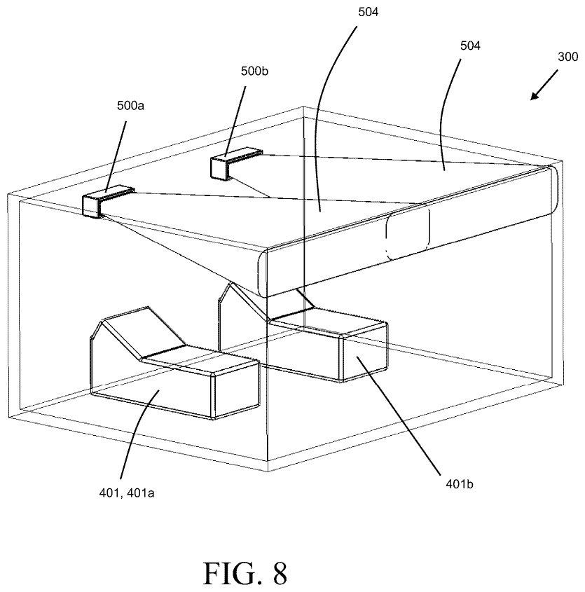

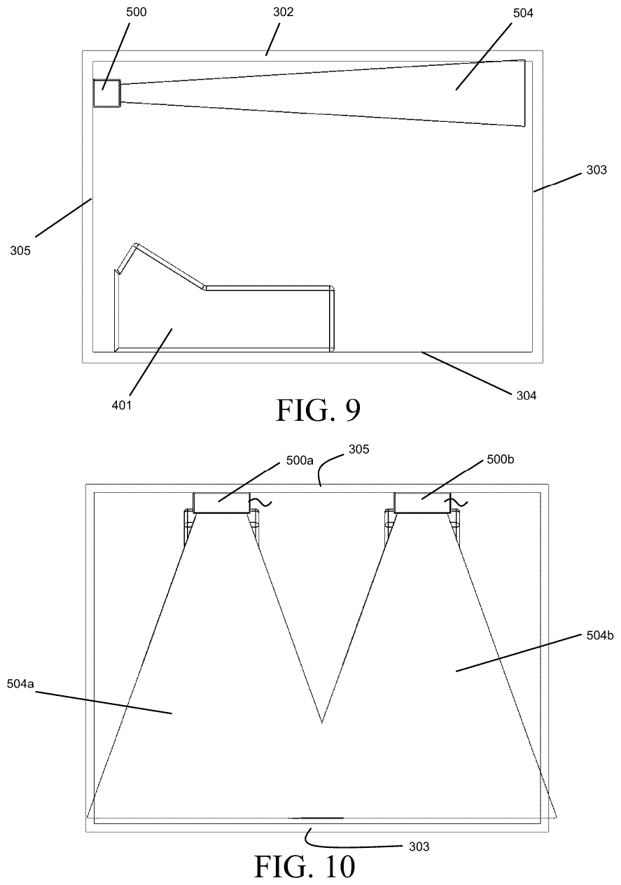

Referring to FIGS. 8-10, an exemplary room 300 includes ceiling 302, floor 304, first, e.g., rear, wall 305, and opposite, e.g., front, wall 303. One or more hospital beds 401a and 401b are positioned within the room. Upper-air luminaires 500a and 500b are mounted near ceiling 302, such as for instance upon rear wall 305. Ultraviolet radiation field 504 is generally directed above head-level, i.e., at least seven feet above floor 304, to ensure that occupants within room 300 are not exposed to the harmful ultraviolet radiation as shown in FIG. 9 which provides a side elevation view of room 300 illustrating ultraviolet radiation field 504 relative to hospital bed 401. FIG. 10 illustrates a top plan view of room 300 showing ultraviolet radiation zones 504a and 504b generated by luminaires 500a and 500b.

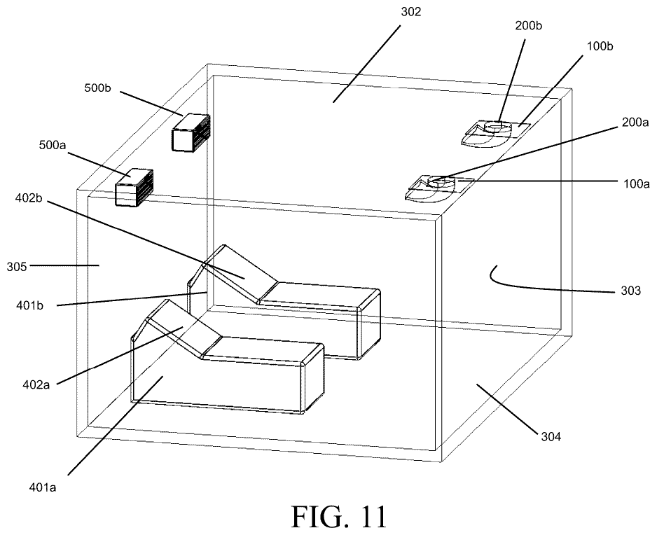

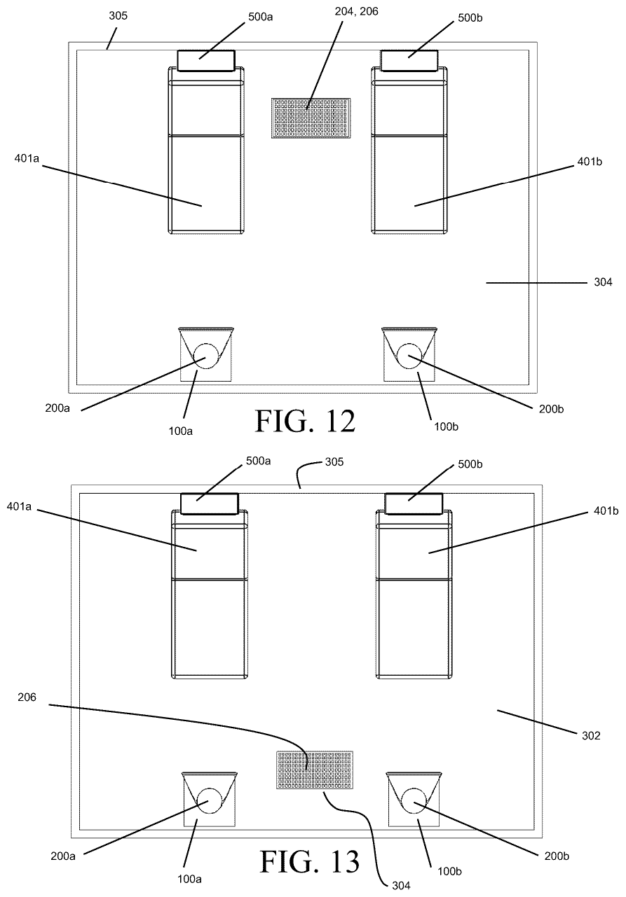

Turning now to FIGS. 11-13, exemplary hospital room 300 has been equipped with diffuser assemblies 100a and 100b relative to HVAC supply outlets 200a and 200b, hospital beds 401a and 401b, and occupant locations 402a and 402b. FIGS. 12 and 13 illustrate alternative HVAC return configurations including HVAC return grille 204 and return duct 206, demonstrating flexibility of diffuser placement relative to room geometry. In FIG. 12, the return air will have traversed the ultraviolet zone reducing active pathogens. This configuration may be preferred if the HVAC system recycles interior air. In FIG. 13, the return air has not yet transversed the ultraviolet zone so a higher concentration of active pathogens exists than the configuration of FIG. 12. This configuration may be preferred if return air is exhausted outside.

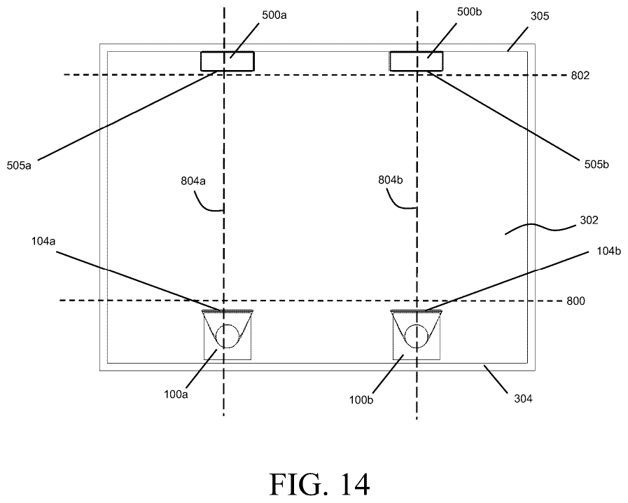

Referring to FIG. 14, diffuser outlet apertures 104a and 104b are arranged along diffuser alignment axis 800, which is substantially parallel to luminaire emission apertures 505a and 505b arranged along luminaire alignment axis 802. This coordinated alignment promotes efficient entrainment of room air into upper ultraviolet regions. Additionally, and without limitation thereto, each pair of diffuser outlet apertures 104/luminaire emission apertures 505 may also be generally arranged along a common axis 804a, 804b whereby air exiting diffuser outlet apertures 104 coincides with the ultraviolet radiation emitted from luminaires 500.

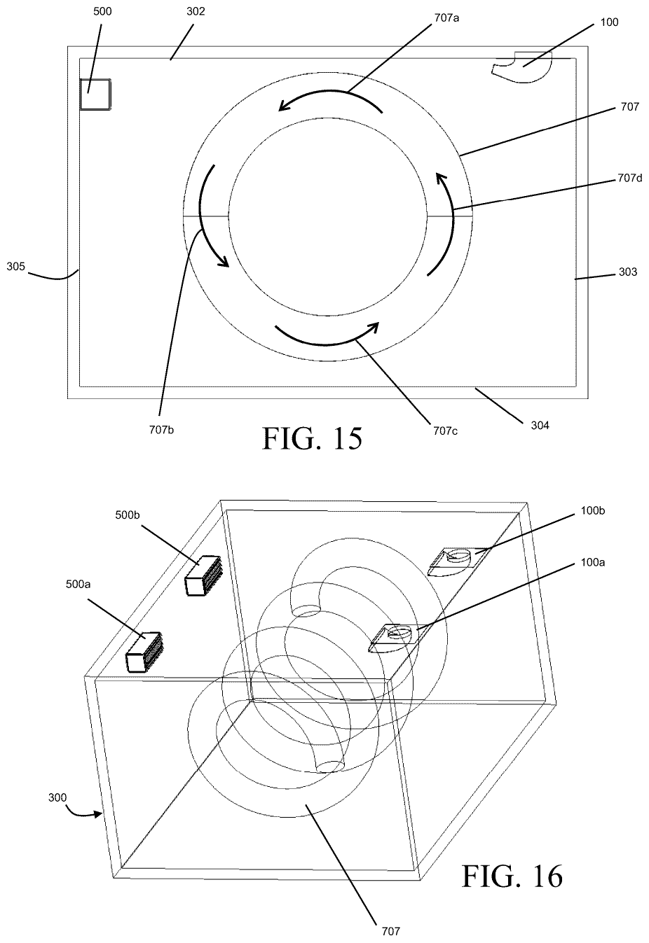

FIGS. 15 and 16 illustrate representative rotational airflow column/loop 707 established within room 300. Airflow portion 707a along ceiling 302 adheres to ceiling 302 via the Coandă effect before airflow portion 707b descends along first wall 305 whereby airflow portion 707c sweeps floor 304 gathering contaminants before airflow portion 707d rises along opposite wall 303 and returns toward ceiling 302 to complete continuous circulation loop 707.

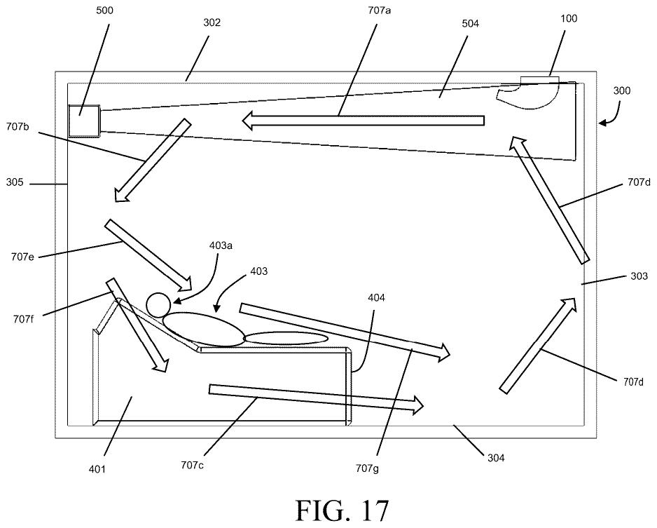

FIG. 17 further illustrates interaction of rotational airflow loop 707 with an occupant 403 positioned on hospital bed 400. Clean airflow portion 707e is delivered toward the occupant breathing zone 403a while pathogen-laden airflow portion 707g, such as exhalations by occupant 403, is entrained into the circulating flow 707 and transported downward toward airflow portion 707c along floor 304. The combined airflow portions 707c, 707g sweep along floor 304 where higher pathogen concentrations are found due to settling. Airflow portion 707d then transports the pathogen concentrated air up into the ultraviolet zone for enhanced pathogen inactivation over prior art technology systems.

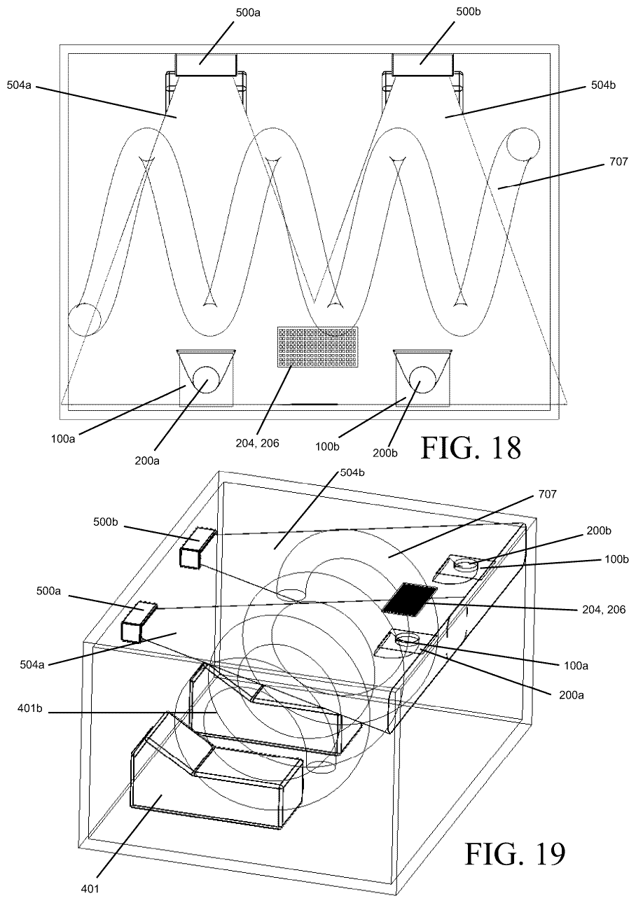

FIGS. 18 and 19 illustrate repeated ultraviolet exposure achieved through recirculating airflow paths 707 passing through ultraviolet irradiation zones 504a and 504b generated by luminaires 500a and 500b.

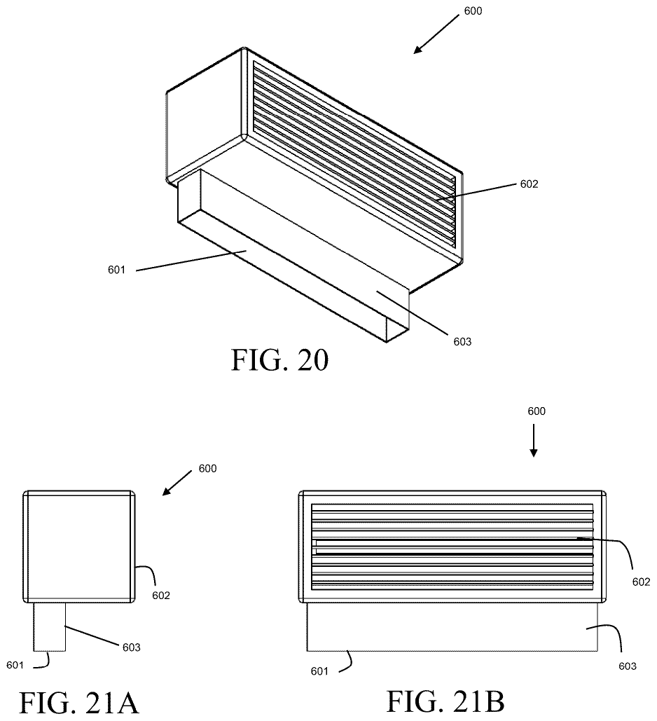

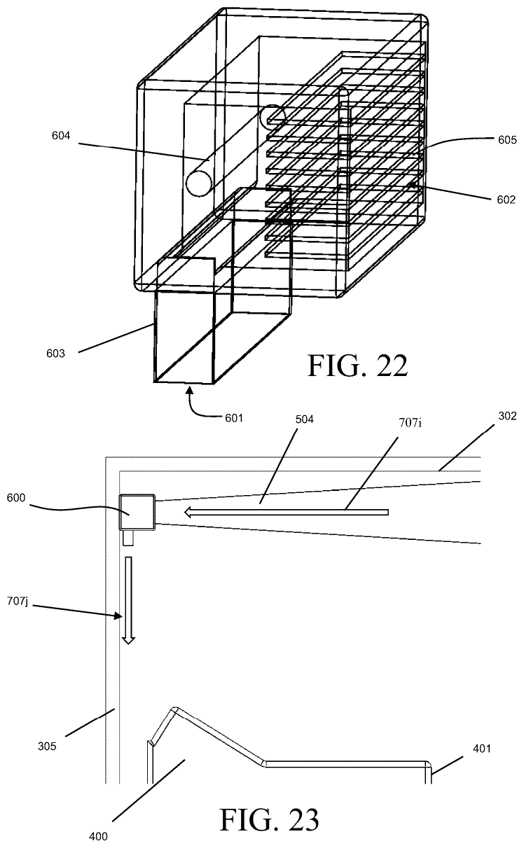

Referring to FIGS. 20-23, an alternative embodiment includes an airflow-integrated ultraviolet luminaire 600 having airflow inlet 602, airflow outlet 601, ultraviolet source 604, light-blocking outlet 603, and internal light-blocking baffles 605. In this embodiment, ceiling-attached airflow is received directly into luminaire 600 for enhanced ultraviolet exposure prior to re-entry into the room. As shown most clearly in FIG. 23 the interaction between rotational airflow 707 and airflow-integrated luminaire 600 allows airflow inlet 602 to direct at least a portion of airflow portion 707a to enter luminaire 600 as airflow portion 707i. Airflow portion 707i is then exposed to concentrated radiation from ultraviolet source 604 before exiting as airflow portion 707j to couple with airflow portion 707b.

In operation, exemplary systems may utilize existing HVAC airflow redirected by diffuser assemblies 100 to establish a room-scale rotational airflow column that entrains contaminated lower air and repeatedly delivers it into upper ultraviolet regions, thereby enhancing pathogen inactivation while simultaneously improving air circulation for occupants. The flow past occupants solves a “reinfecting” problem experienced by patients with severe respiratory infections and extremely shallow breathing ability. Heavily pathogen-concentrated air exiting an occupant is pushed away and the highest air quality air in the room circulates from above and behind for the occupants'next breath. Attending staff working with the occupant standing on either side of the bed also receive the cleanest air in the room that is flowing down first wall 305 as shown as clean airflow portion 707e in FIG. 17. Staff and visitors may be cautioned from standing at or near end/foot 404 of the bed where air contaminated by the occupant is traveling as shown by 707g in FIG. 17.

It should be noted by those skilled in the art that diffuser 101 orientation may be selected based on room geometry, bed placement, HVAC supply and return locations, and computational fluid dynamics modeling, enabling flexible deployment across a wide range of retrofit and new-construction environments. It should be further noted that the exemplary room configuration described herein is one of many alterative rooms such as medical exam rooms, classrooms, lecture halls, conference rooms etc. that would benefit from the rotational airflow technology the diffuser 100 enables.

This disclosure has been described in detail with particular reference to an embodiment, but it will be understood that variations and modifications can be affected within the spirit and scope of the disclosure. The presently disclosed embodiments are therefore considered in all respects to be illustrative and not restrictive. The scope of the invention is indicated by the appended claims, and all changes that come within the meaning and range of equivalents thereof are intended to be embraced therein.

What is claimed is:

1. A system for enhancing ultraviolet air disinfection in an enclosed space, wherein the enclosed spaced is coupled to an HVAC supply configured to deliver conditioned air into the enclosed space, and wherein the enclosed space is defined by an upper region proximate a ceiling, a lower region proximate a floor, and a occupation region intermediate the upper region and the lower region of the enclosed space, the system comprising:
a) at least one Coandă-effect diffuser configured to be coupled to the HVAC supply and mounted adjacent the ceiling of the enclosed space, the diffuser configured to redirect supplied air received from the HVAC supply along the ceiling to induce a room-scale rotational airflow pattern; and
b) at least one upper-air ultraviolet luminaire positioned within the upper region of the enclosed space and configured to generate an ultraviolet irradiation region;
wherein the diffuser is configured to establish a circulating airflow that entrains contaminated air from the lower region and transports the contaminated air into the ultraviolet irradiation region produced by the upper-air ultraviolet luminaire; and
wherein the circulating airflow delivers treated air from the ultraviolet irradiation region toward the occupation region of the enclosed space.

2. The system of claim 1, wherein the diffuser defines substantially equal inlet and outlet flow cross-sectional areas.

3. The system of claim 1, wherein airflow exits the diffuser at approximately the same average velocity as airflow entering the diffuser.

4. The system of claim 1, wherein the diffuser redirects airflow through an average directional change exceeding approximately 90 degrees.

5. The system of claim 1, wherein an outlet aperture defined by the diffuser is arranged along an axis substantially parallel to an axis defined by an emission aperture defined by the upper-air ultraviolet luminaire.

6. The system of claim 1, wherein an outlet aperture defined by the diffuser is arranged at an angle relative to the plane defined by the ceiling of enclosed space.

7. The system of claim 1, wherein the angle of the outlet aperture defined by the diffuser relative to the plane defined by the ceiling of enclosed space is between about zero degrees and about twenty-five degrees.

8. The system of claim 1, wherein a portion of the circulating airflow is directed toward a breathing zone of an occupant within the occupation region.

9. The system of claim 1, wherein diffuser orientation is selected based on computational fluid dynamics modeling accounting for one or more of room geometry, occupant location, HVAC supply placement, and return grille placement.

10. The system of claim 1, wherein the diffuser is configured for retrofit installation onto an existing HVAC ceiling outlet.

11. The system of claim 1, wherein the at least one upper-air ultraviolet luminaire is an airflow-integrated ultraviolet luminaire configured to receive treated air from the ultraviolet irradiation region and expose the treated air to ultraviolet radiation within the airflow-integrated ultraviolet luminaire prior to re-entry of the treated air into the circulating airflow.

12. The system of claim 11, wherein the airflow-integrated ultraviolet luminaire includes internal light-blocking baffles.

13. The system of claim 1, wherein the enclosed space comprises a healthcare room.

14. The system of claim 13, wherein the enclosed space includes at least one hospital bed.

15. The system of claim 1, wherein the enclosed space comprises at least one of a classroom, medical examination room, conference room, auditorium, office, long-term care facility, or residential space.

16. A method of reducing airborne pathogen concentration within an enclosed space, comprising:
a) supplying air into the enclosed space from an HVAC system;
b) redirecting the supplied air along a ceiling using a Coandă-effect diffuser to generate a rotational airflow column within the enclosed space;
c) entraining contaminated air from a lower region of the enclosed space into the rotational airflow column;
d) transporting the contaminated air into an upper ultraviolet irradiation region produced by an upper-air ultraviolet luminaire;
e) repeatedly exposing the air to ultraviolet radiation through continued circulation; and
f) delivering treated air back toward an occupied region of the enclosed space.

17. The method of claim 16, further comprising aligning diffuser outlet apertures of the Coandă-effect diffuser substantially parallel to emission apertures of the upper-air ultraviolet luminaire.

18. The method of claim 16, further comprising selecting a diffuser orientation based on computational fluid dynamics modeling of one or more of room geometry, occupant location, HVAC supply placement, and return grille placement.

19. The method of claim 16, wherein repeatedly exposing the air to ultraviolet radiation comprises passing the air through the ultraviolet irradiation region multiple times during continuous circulation.

20. The method of claim 16, further comprising directing a portion of the treated airflow toward an occupant breathing zone.
